(19) United States
(12) Patent Application Publication
(10) Pub. No.: US 2005/0158222 A1
(43) Pub. Date: Jul. 21, 2005

(54) Method of manufacture and apparatus for sterilization cassettes and baskets

(75) Inventors: Cary A. Bettenhausen, Indianapolis, IN (US); Tood E. Bettenhausen, Indianapolis, IN (US)

Correspondence Address: Frank D. Lachenmaier;Suite 200, 116 N. Main St, P.O. Box 1047, Kokomo, IN, 46903-1047, US

(73) Assignees: William I. Summers, Kokomo, IN (US); Connie L. Summers, Kokomo, IN (US)

(21) Appl. No.: 11/037,675

(22) Filed: Jan. 18, 2005

Related U.S. Application Data
(60) Provisional application No. 60/536,656, filed on Jan. 15, 2004.

(51) Int. Cl. A61L 2/26

(52) U.S. Cl. 422/300; 29/592

(57) ABSTRACT

In accordance with the present invention a tray for sterilizing, storing, transporting and presenting surgical instruments is made by drawing or hydroforming a light weight, malleable sheet metal into die cavities with the shapes of the tools in question and in some cases a basket shape to hold several small instruments such as screws with no sharp edges or burrs to puncture or tear the sterile gauze wrap and a high heat transfer coefficient to expedite the drying process after sterilization.

### BACKGROUND

1. Field of Invention

This invention relates generally to materials handling apparatus and an improved method of manufacture of same. More specifically, this invention relates to an integrated system of pockets and baskets for facilitating sterilization, storage and presentation of surgical instruments.

2. Prior Art

A variety of sterilization cassettes exist with different shapes and styles of pockets and retainers to hold various surgical instruments securely during sterilization. Where possible, some surgical instruments are single use, arriving from the manufacture in a sterile package to be opened during surgery and then thrown away. There are many instruments used in modern surgery, however, that are delicate, complex and expensive and must be sterilized and re-used. A common practice is also to present all of the instruments to be used in a given surgery to the surgeon in a common cassette or carrier. Holding the instruments securely during sterilization and transport is usually accomplished by trapping them between a deformable member with sterilant ingress ports and a top enclosure also with mating sterilant ingress ports. Once a pre-arranged cassette is loaded with the correct instruments for the desired surgery and sterilized, the cassette is typically wrapped in a sterile gauze material and stored until required transportation to the next operating room. It is unwrapped, the top enclosure and deformable member removed and presented to the surgical team. These sterilization trays suffer from handling damage and relatively quick obsolescence and are expensive to maintain.

The myriad of such cassettes or carriers can be broken into two major types each with their own advantages and drawbacks. The first and earliest of these were sheet metal cases with brackets riveted into holes stamped onto the bottom of the case and weep ports punched through the bottom of the sheet metal case to allow for egress of the sterilizing condensate. Side walls were formed up from the bottom blank, overlapped and riveted to form a secure lower box. An example of this type is shown in FIG. 1 Prior Art. The brackets were sized to fit the instruments required for a given operation. The sheet metal case had a relatively high coefficient of heat transfer that allowed fairly rapid drying, even though the drying cycle was still the longest segment of the sterilization process. Rapid drying time may be critical if an instrument is dropped during an operation and requires re-sterilization before the operation can be completed. Some kits compensate for this possibility by including redundant instruments within the kit at the obvious extra cost.

The two biggest drawbacks to the stamped sheet metal case are the inherent weight and the fact that it very difficult and expensive to punch holes in sheet metal with conventional punch and die clearance leaving no burrs or sharp edges around the openings or edges of the case. As mentioned above, once the case full of instruments has been sterilized, it is desirable to have it wrapped in a sterile gauze and stored awaiting transport to an operating room and presentation to the surgical team. Any sharp edges or comers can pierce the sterile gauze and require re-sterilization before use. Some instruments are fairly large and heavy and their packaging is limited by the weight that the technician can handle safely.

A number of cassettes have been fabricated from high temperature plastics molded or vacuum formed in an attempt to reduce these two problems. The plastic cases are inherently lighter in weight and can be molded essentially burr and sharp edge free. An example of this type is shown in FIG. 2 Prior art.

Continued exposure to high temperatures and humidity, essentially an autoclave environment, can cause deterioration and fracturing of some plastics over time. The biggest problem with the plastic cases, however, is their relatively low coefficient of heat transfer compared to metal cases. The drying time is dramatically impacted by the sterilization case's ability to conduct the heat away from the instruments.

### SUMMARY

An object of the present invention is to provide a case for sterilizing, storing, transporting and presenting surgical instruments of different sizes and shapes in a light weight cassette formed with no sharp edges and from a material with high heat transfer coefficient. Another object of the present invention is to provide a cassette which retains the instruments securely during processing, but allows the surgical team to remove them readily when required. A further object is for the instrument retaining cassette to be economical to revise or replace, facilitating different instruments when the procedure changes, the sequence of presentation changes or the case is damaged in handling. The case should also contain basket shaped pockets to hold small instruments such as screws. In the preferred embodiment the case is made by drawing or hydroforming a malleable sheet of metal into die cavities with either the shape of the tool in question and/or a basket shape to hold several small instruments.

Individual die cavity blocks are made with standard exterior dimensions that can be assembled into desired combinations and arrangements within a standard die shoe prior to the drawing or hydro forming operation. The die blocks have unique interior dimensions to pocket the instruments in question in the arrangement most efficient for the surgical team's demands. If a new instrument or sequence of instruments is desired a new die cavity can be made and substituted into the die shoe or the blocks rearranged to the new sequence and a new sheet formed to replace the obsolete sterilization tray.

### DRAWINGS

In order that the invention may be more fully understood it will now be described by way of example, with reference to the accompanying drawings in which:

FIG. 1 is a front perspective view of a Prior Art stamped sheet metal case with riveted brackets.

FIG. 2 is a front perspective view of a Prior Art molded plastic sterilization tray.

FIG. 3 is a front perspective view of a Hydro-formed metal sterilization tray with various components utilized for the several applications shown in phantom lines, not part of this invention.

### REFERENCE NUMERALS

The same reference numbers are used to refer to the same or similar parts in the various views.

10—Instrument sterilization case

12—pockets

14—instruments

16—weep or egress ports

18—brackets

20—top enclosure

2—deformable member

24—filter

26—ingress ports

### DESCRIPTION

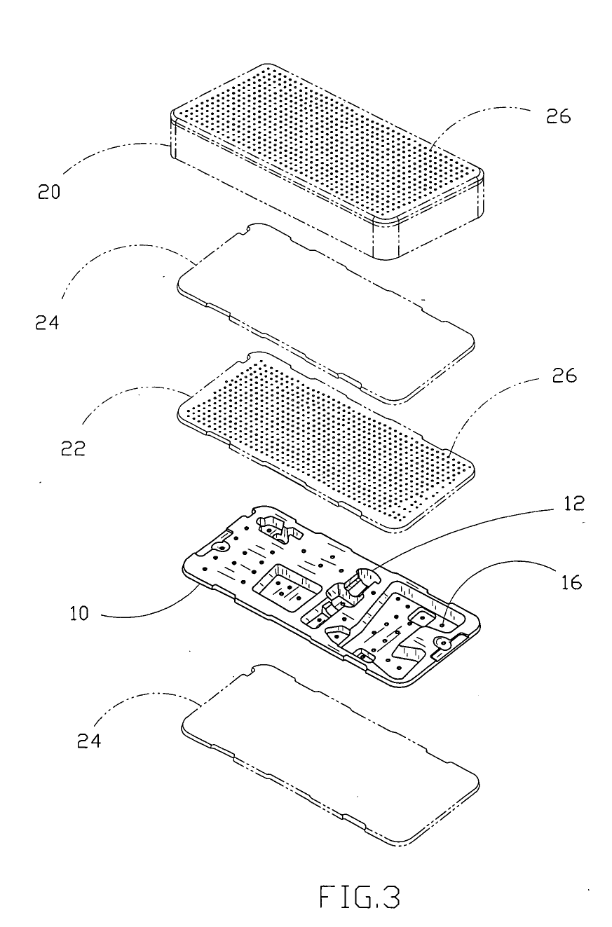

In order that the invention may be more fully understood, it will now be described by way of example with reference to the accompanying drawings in which FIG. 3 illustrates an instrument sterilization tray 10 with various shaped pockets 12 to match outlines of instruments 14.

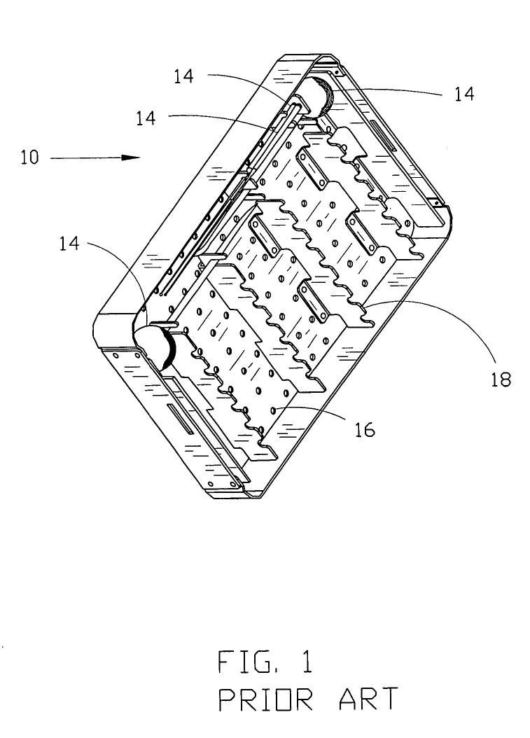

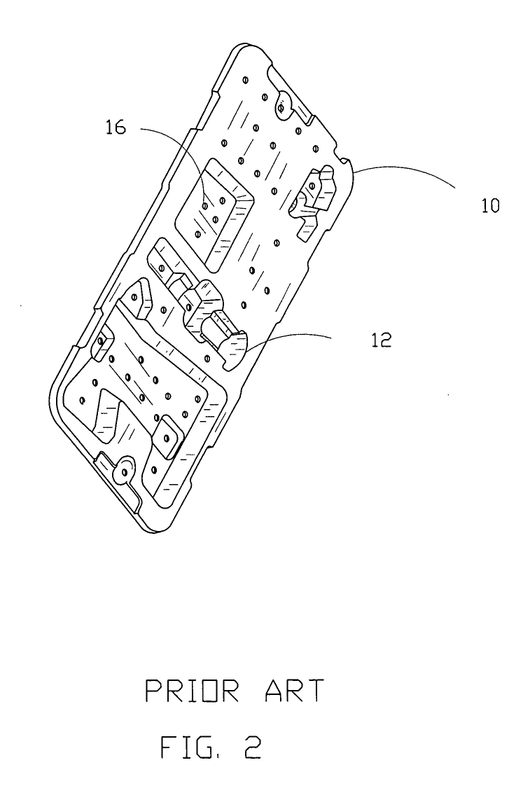

The improvement in the method of manufacture involves replacing the stamped sheet metal case with riveted brackets as shown in FIG. 1 or the molded plastic case as shown in FIG. 2 with a drawn or hydro-formed metal sheet case shown in FIG. 3. The blank is developed by one skilled in that art sufficiently larger than the final outline such that there is enough material to be drawn into the die cavities without tearing at the corners. It is formed down on the outer periphery creating side walls which provide added strength and means to attach to top enclosure 20 (shown in phantom lines and not part of this invention). Weep ports 16 in the bottoms of pockets 12 can be added either in the blank sheet or punched or drilled post-drawing. The drawing or hydro-forming process, with properly designed dies, generates a pocketed case 10 with similar blending of corners and elimination of burrs as is attainable with a plastic molded case, but with the significant benefits of the durability, strength and most importantly heat transfer coefficient of a stamped sheet metal case.

If multiple instruments 14 that have small difficult to recognize detail differences are required for the surgery in question, a mirror image of a descriptor word or number can be embossed on the surface of the die block cavity adjacent to the pocket that will transfer the word or number to the formed sheet to assist in identifying the proper tool for the appropriate stage of the procedure.

Instruments are retained in the case with a deformable member 22 (shown in phantom lines and not part of this invention) with matching ports 26 to allow ingress of the sterilizing fluid, laid over pocketed instruments 14, restrained by top enclosure 20 (shown in phantom lines and not part of this invention) with matching ingress holes 26. Top enclosure 20 (shown in phantom lines and not part of this invention) can be fastened to formed case 10 with conventional means during sterilization, wrapping in sterile gauze, storage and transport, being removed after the sterile gauze has been removed in the operating room. Deformable member 22 is then removed, exposing the instruments for use in the surgical procedure.

Formed case 10 may also be used in a filtered case with out top enclosure 20 or as a filtered case with filters 24 mounted on the bottom of case 10 and/or on a gasketed top enclosure 20, shown for illustration purposes only in phantom lines in FIG. 3.

### OPERATION

Die cavity blocks are formed by conventional machining or electro discharge machining (EDM) from conventional tool steels in blocks where the exterior dimensions are common and interior dimensions match the contour of instruments 14 in question or form baskets for small parts such as screws. These die cavity blocks are then assembled into a rectangular shaped die shoe and mounted in either a drawing press or a hydro-forming machine and a properly oversized sheet of malleable metal is placed on top of the die cavity assembly and formed. Weep ports 16 that are required to allow the condensing sterilization fluid to escape as case 10 cools can be placed in the blank before forming or punched or drilled in the bottom of pockets 12 post-forming.

The metal sheet formed into case 10 in this manner then has the benefits of the additional strength and durability of formed metal over plastic. It also has the benefit of the speed of drying that is inherent from metal's superior heat transfer capability. The drawn or hydro-formed tray enjoys the smooth shape of the molded plastic tray, thus not jeopardizing the integrity of the sterile muslin gauze wrap.

A second embodiment would utilize a sheet of a heavy wire screen of a malleable material that allows the sterilant condensate to escape through the screen mesh, avoiding the need to construct weep ports in the bases of the pockets.

The preceding descriptions are for illustrative purposes and are not intended to limit the scope of this invention. The scope of the invention should be determined by the appended claims rather than by the specific examples given. Manufacturing techniques described herein are well known to those skilled in the arts of mass production but have not been embraced for this particular application and hold forth significant benefits for improving the cost effectiveness of these critical and rapidly escalating costs of modern surgery.

1. In a sterilization tray assembly for sterilizing, transporting and storing surgical instruments comprising a top enclosure with a plurality of ports for allowing the ingress of a gaseous sterilant; a pocketed case with said pockets sized to accept said instruments required for a particular surgical procedure with weep ports formed in the bottom of said pockets to allow the egress of sterilant condensate; a means for locking said top enclosure to said case; cooperating deformable members fitting in said top enclosure for supporting and clamping said instruments in said pockets where said deformable member has matching ports to allow said sterilant to reach said instruments; and said case assembly is wrapped in sterile gauze after sterilization for storage and transport,
the improvement which comprises utilizing a case fabrication process selected from either deep drawing or hydro-forming, whereby said case is formed, without the sharp corners or burrs that are common with sheet metal stamping that might puncture or tear said sterile gauze, from a blank of malleable light weight metal that has a higher heat transfer coefficient than plastics whereby allowing a faster drying time than is available with plastic molded cases.

2. In a sterilization tray assembly for sterilizing, transporting and storing surgical instruments according to claim 1 wherein said blank of light weight malleable metal is formed from a sheet of a metal mesh of heavy screen wire whereby said sterilant condensation is allowed to egress through said screen wire mesh without the need to punch said weep ports in the bottoms of said pockets.

3. In a sterilization tray assembly for sterilizing, transporting and storing surgical instruments comprising a filter case with top and bottom filters; a plurality of ports for allowing the ingress of a gaseous sterilant; a pocketed case with said pockets sized to accept said instruments required for a particular surgical procedure with weep ports formed in the bottom of said pockets to allow the egress of sterilant condensate; cooperating deformable members fitting on top of said pocketed case for supporting and clamping said instruments in said pockets where said deformable member has matching ingress ports to allow said sterilant to reach said instruments; a means for locking said deformable member to said case; and said case assembly is wrapped in sterile gauze after sterilization for storage and transport,
the improvement which comprises utilizing a tray fabrication process selected from either deep drawing or hydro-forming whereby the tray assembly is formed without the sharp corners or burrs that are common with sheet metal stamping that might puncture or tear said sterile gauze; from a blank of malleable light weight metal that has a higher heat transfer coefficient than plastics which allows for a faster drying time than is available in plastic molded cases.

4. In a sterilization tray assembly for sterilizing, transporting and storing surgical instruments according to claim 3 wherein said blank of light weight malleable metal is formed from a sheet of a metal mesh of heavy screen wire whereby said sterilant condensation is allowed to egress through said screen wire mesh without the need to punch said weep ports in the bottoms of said pockets.

5. In a sterilization tray assembly for sterilizing, transporting and storing surgical instruments comprising a top enclosure with a filter; a plurality of ports for allowing the ingress of a gaseous sterilant; a pocketed case with said pockets sized to accept said instruments required for a particular surgical procedure with weep ports formed in the bottom of said pockets to allow the egress of sterilant condensate; cooperating deformable members fitting on top of said pocketed case for supporting and clamping said instruments in said pockets where said deformable member has matching ports to allow said sterilant to reach said instruments; a means for locking said top enclosure to said case; and said case assembly is wrapped in sterile gauze after sterilization for storage and transport,
the improvement which comprises utilizing a case fabrication process selected from either deep drawing or hydro-forming whereby the case assembly is formed without the sharp corners or burrs that are common with sheet metal stamping that might puncture or tear said sterile gauze; from a blank of malleable light weight metal that has a higher heat transfer coefficient than plastics which allows for a faster drying time than is available in plastic molded cases.

6. In a sterilization tray assembly for sterilizing, transporting and storing surgical instruments according to claim 5 wherein an additional filter is placed on the underside of said case.

7. In a sterilization tray assembly for sterilizing, transporting and storing surgical instruments according to claim 6 wherein said blank of light weight malleable metal is formed from a sheet of a metal mesh of heavy screen wire whereby said sterilant condensation is allowed to egress through said screen wire mesh without the need to punch said weep ports in the bottoms of said pockets.

8. In a sterilization tray assembly for sterilizing, transporting and storing surgical instruments according to claim 5 wherein said blank of light weight malleable metal is formed from a sheet of a metal mesh of heavy screen wire whereby said sterilant condensation is allowed to egress through said screen wire mesh without the need to punch said weep ports in the bottoms of said pockets.
